(19) United States
(12) Patent Application Publication
(10) Pub. No.: US 2011/0098621 A1
(43) Pub. Date: Apr. 28, 2011

(54) TRANSPARENT FILM DRESSING

(75) Inventors: Tomas Fabo, Molnlycke (SE); Anna Svensby, Vastra Frolunda (SE)

(73) Assignee: MOLNLYCKE HEALTH CARE AB, Goteborg (SE)

(21) Appl. No.: 12/741,646

(22) PCT Filed: Dec. 8, 2008
(86) PCT No.: PCT/SE2008/051419

§ 371 Date: Jul. 22, 2010

(30) Foreign Application Priority Data
Dec. 18, 2007 (SE) 0702811-1

(51) Int. Cl. A61F 15/00

(52) U.S. Cl. 602/52

(57) ABSTRACT

A transparent film dressing (1) for protection of skin surrounding a wound, the dressing includes a film layer (2) coated with a skin friendly adhesive (3) on one side, a stiffening layer (4) releasably attached to the film layer on the side thereof opposite to the side coated with adhesive, and a release layer (5) releasably attached to the adhesive coating on the film layer. A protection layer (6) is releasably attached to the release layer (5) on the side thereof opposite to the side attached to the adhesive coating (3), and a hole (9) extends through the stiffening layer (4), the coated film layer (2,3) and the release layer (5) but not through the protection layer, all the layers being transparent. A method of applying such a dressing is also described.

### TECHNICAL FIELD

The present invention relates to a transparent film dressing for protection of skin surrounding a wound, said dressing comprising a film layer coated with a skin friendly adhesive on one side, a stiffening layer releasably attached to the film layer on the side thereof opposite to the side coated with adhesive, and a release layer releasably attached to the adhesive coating on the film layer. The invention also relates to a method of applying such a dressing.

### BACKGROUND OF THE INVENTION

One of the most important functions possessed by human skin is to constitute the body's barrier to the environment. The skin protects against the harmful effects of microorganisms, toxic substances, heat, cold, mechanical damage, etc. The skin also constitutes a necessary protection against dehydration. The skin around wounds, in particular what are termed chronic wounds, which take anything from weeks to months to heal, is frequently in a worse condition than the remaining skin and its barrier function is consequently impaired. There can be several reasons for this circumstance. Some wounds originate in the main from underlying diseases which give rise to locally impaired circulation and nutrition, thereby weakening the skin and making it more easily damaged. Many wounds are moist and produce exudate which frequently leaks out onto the skin around the wound, with the moisture causing an impaired skin barrier function so that the skin begins to disintegrate. The wound exudate contains enzymes, microorganisms and other substances which can have a harmful effect on the skin, particularly on disintegrating skin. Dressings which are used on wounds are frequently provided with self-adhesive glue. The purpose of the self-adhesive glue is for it to adhere to the skin around the wound and fix the dressing at the intended site. However, a disadvantage of self-adhesive glue is that the skin can be damaged when the dressing is removed and that disagreeable pain can simultaneously ensue. It is especially troublesome when the self-adhesive dressings have to be changed frequently over a relatively long period. Systemic or local treatment with cortisone, radiation, cell poisons or other medical preparations can weaken the skin still further.

The most frequently employed method of protecting the skin around wounds is to lubricate skin with a protective ointment, cream or paste. Ordinary paraffin, e.g. Vaseline, is sometimes employed. Other frequently occurring preparations, which are often termed “barrier creams” in English usage, are ACO zinc paste (ACO hud AB [ACO Skin AB], Upplands Väsby, Sweden), Baza® Protect from Coloplast (Coloplast Corp. Marietta, Ga., USA), 3M™ Durable Barrier Cream (3M Health Care, St. Paul, Minn., USA), Inotyol (Laboratories URGO, Dijon, France) and Silon (Smith & Nephew AB, Mölndal, Sweden). A barrier cream increases the resistance of the skin to liquid and other harmful substances which come to be on the skin. The protective ointment/cream/paste layer prevents the wound liquid from coming into direct contact with the skin. When weeping wounds are bandaged, use is frequently made of a relatively thick, approx. 1 cm-wide, layer of a highly viscous ointment or paste (for example zinc paste) on the skin closest to the wound. A thinner layer of a moisture-preserving, protective ointment or cream is applied outside this strand in order to prevent the skin from drying out and thereby improve the intrinsic barrier function of the skin.

When ointments/creams/pastes are used around wounds, they also have a function in addition to protecting the covered skin. The ointment/cream/paste prevents peripheral leakage of wound liquid from the wound to the skin outside the wound at the same time as it protects against passage of liquids, for example urine, from the outside and inside the wound.

Many preparations comprise active components such as hydrocortisone, urea, ZnO and antimicrobial substances which reduce the irritation of the skin and/or facilitate healing of the skin. The ointment or cream is sometimes applied by means of what is termed an ointment compress, which is a more or less sparse textile material which is impregnated with an ointment of the above mentioned type. The compress is laid over the region of the wound such that it extends for some distance over the skin.

The abovementioned ointments, creams and pastes suffer from several disadvantages. They possess very low cohesion and are therefore felt to be sticky and are frequently difficult to keep in place under a dressing since they do not have any dimensional stability but instead behave as viscous liquids. They can leak into the wound, be absorbed in the wound dressing or leak out from the region of the wound and soil clothes, etc. Self-adhesive dressings cannot be fixed to skin which is coated with these preparations since the adhesion is inactivated. As a result, leakage of wound liquid between the skin and the dressing frequently occurs. Wiping off old paste and ointment is frequently a time-consuming step when changing dressings.

Another method is to protect the skin by applying, to the skin around a wound, a liquid which contains a solid substance which is dissolved or dispersed in a volatile liquid. U.S. Pat. No. 5,741,509 provides an example of such a method. Following application to the skin, the volatile substance evaporates off and leaves behind a protective film of the solid substance. The liquid can be applied in spray form or by being spread with a cotton wad, for example. An example of this type of product is 3M™ Cavilon™ No Sting Barrier Film (3M Health Care, St. Paul, Minn., USA).

A disadvantage of this method is that it is difficult to remove the protective film from the skin and it is also difficult to obtain a sufficiently thick layer of the protective material; as a result, the method is not always sufficiently effective. Nor does this type of product adequately prevent peripheral leakage of wound liquid from the wound or passage of urine, etc., into the wound from the exterior.

Skin-protecting ointments/pastes also have an important function in situations other than those connected with caring for the skin around wounds. For example, sensitive and damaged skin is also found around different types of stomata and where the skin is penetrated with different types of tubes. Leakage of more or less aggressive body liquids frequently occurs in connection with these applications and the skin is subjected to frequent changes of self-adhesive dressings. While ointments are often used in this connection, they can be problematical to employ when they prevent adhesion of stoma bags or other articles which may need to be attached.

The objective of the present invention is to provide a device for protection of skin surrounding a wound which device is easy to apply and remove lacking the disadvantages of the protective devices according to prior art.

### SUMMARY OF THE INVENTION

This objective is accomplished by transparent film dressing for protection of skin surrounding a wound, said dressing comprising a film layer coated with a skin friendly adhesive on one side, a stiffening layer releasably attached to the film layer on the side thereof opposite to the side coated with adhesive, and a release layer releasably attached to the adhesive coating on the film layer, characterised in that a protection layer is releasably attached to the release layer on the side thereof opposite to the side attached to the adhesive coating, and in that a hole extends through the stiffening layer, the coated film layer and the release layer but not through the protection layer, all said layers being transparent. Since all layers of the dressing are transparent it is possible to visualize the contour of a wound when the dressing is placed over a wound. This contour can then be marked on the stiffening layer. The protection layer protects the remaining components of the dressing from being soiled by exudate from the wound bed while marking the stiffening layer. The marking of the contour of the wound bed on the stiffening layer makes it possible to accurately adapt the dressing to cover all skin surrounding the wound by cutting the dressing along the marked contour. The hole in the dressing facilitates such cutting with the help of scissors. The skin friendly adhesive and the thin film of the dressing ensures that liquid transport underneath the dressing from the wound bed or to the wound bed is prevented and the stiffening layer makes it easy to accurately match the inner contour of the dressing with the outer contour of the wound bed during application of the dressing. The skin friendly adhesive makes it possible to change the dressing without damage to the skin.

In a preferred embodiment, the protection layer at least in part or parts extends beyond the contour of the other layers of the dressing, and is attached to the release layer by one or more seams, for example by one seam extending between opposite edges of the release layer without reaching said edges, said seam being located in an end part of the release layer but distanced from an adjacent end edge. Each seam can be continuous or discontinuous and constituted by a string of adhesive or by a weld seam.

The protection layer is preferably a plastic film and the release layer is preferably a plastic film having a pattern of protrusions on each of its sides.

The stiffening layer can to advantage be divided into a frame portion and a central portion by a cutting line.

The invention also relates to a method of applying such a film dressing to the skin surrounding a wound comprising the following steps;
applying the dressing over a wound so that the wound bed and an area surrounding the wound is covered by the film layer of the dressing and so that the hole in the dressing is located over the wound bed,
marking the contour of the wound bed on the stiffening layer,
removing the dressing from the wound,
removing the protection layer from the dressing,
cutting an opening in the remaining parts of the dressing corresponding to the marked contour of the wound bed, whereby the cutting is facilitated by the hole in the dressing,
removing the cut part of the dressing,
removing the release layer,
applying the remaining part of the dressing over the wound so that the edges of the opening cut therein correspond with the contour of the wound, and
removing the stiffening layer from the film layer.

If the stiffening layer is divided into a frame portion and a central portion by a cutting line the remaining part of the central portion of the stiffening layer is removed before the step of applying the remaining part of the dressing over the wound.

The cut out portion of the stiffening layer or the portion of the stiffening layer remaining after the cutting out of a portion of the dressing corresponding to the size of the wound bed is saved in order to document the size of the wound bed at the time of application of the dressing.

If the stiffening layer is divided into a frame portion and a central portion by a cutting line the cut out part of the central portion of the stiffening layer is saved in order to document the size of the wound bed at the time of application of the dressing.

### BRIEF DESCRIPTION OF THE DRAWINGS

The invention will now be described with reference to the enclosed figures, of which;

FIG. 1 schematically shows, in a plan view, a film dressing according to a first preferred embodiment of the invention,

FIG. 2A shows a cross-sectional view along line II-II in FIG. 1,

FIG. 2B shows a view similar to FIG. 2A of a film dressing according to a second preferred embodiment,

FIG. 3 schematically illustrates the measurement of strength of adhesion to the skin,

FIG. 4 shows a cone used for softness measurement, and

FIG. 5 illustrates a method of measuring softness.

### DESCRIPTION OF EMBODIMENTS

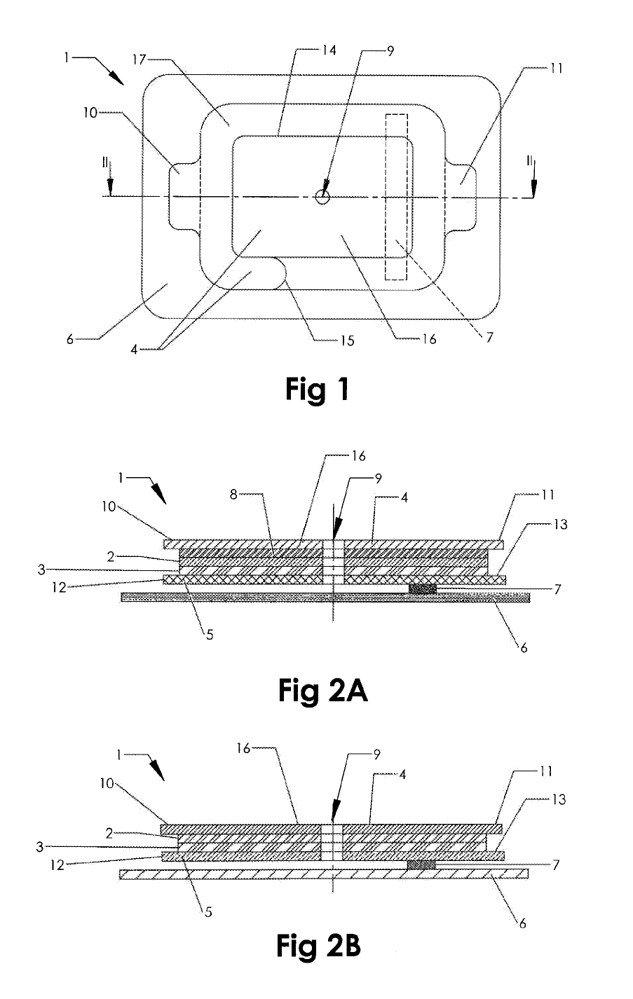

The device for protection of skin surrounding a wound is in accordance with the present invention in essence a transparent film dressing provided with features facilitating such use of the dressing. An embodiment of a transparent film dressing 1 in accordance with a first preferred embodiment the present invention is schematically shown in FIGS. 1 and 2A.

The film dressing 1 includes a film layer 2 and a layer 3 of skin friendly adhesive coated on the lower side thereof. The lower side of the dressing is in this text defined as the side facing the skin in use of the dressing irrespective of the actual position of the skin. The dressing 1 also includes a stiffening layer 4 for facilitate handling and applying of the dressing which is releasably attached to the film layer 2 on the upper side thereof, and a release layer 5 for protecting the adhesive layer 3 during transport and storage before application of the dressing, which is loosely attached to the adhesive layer 3 on the lower side thereof.

The stiffening layer 4 is attached to the film layer 2 by a layer 8 of adhesive.

A protection layer 6 is attached to the release layer 5 by easily opened means, such as glue string 7, and is preferably extended beyond the edges of the release layer 5 around the periphery thereof.

A hole 9 going through all the layers except the protection layer 6 is cut in the middle part of the dressing.

All the components of the dressing 1 are transparent.

The dressing is applied to the skin surrounding a wound in the following way.

Firstly the dressing 1 is applied over a wound so that the wound bed and an area surrounding the wound is covered by the film layer 2 of the dressing and so that the hole 9 in the dressing is located over the wound bed. Since all the layers of the dressing 1 are transparent, the contour of the wound bed can be seen through the dressing and this contour is then carefully marked by a pen or the like on the stiffening layer 4.

After marking of the contour of the wound on the stiffening layer 4, the dressing 1 is removed from the wound.

Thereafter, the protection layer 6 is removed from the dressing. This protection layer will have been soiled by the exudate from the wound but the over-laying layers of the dressing 1, i.e. the release layer 5, the adhesive layer 3, the film layer 2, the adhesive layer 8 and the stiffening layer 4, are all protected from soiling by protection layer 6. By having this layer 6 being significantly larger than the remaining layers in dressing 1, the risk for exudate to soil these layers is minimised. Furthermore, the dimension of the protection layer 6 will almost certainly allow the user to grab an unsoiled part of protection layer 6 when removing this layer from the rest of dressing 1.

After the removal of the protection layer, an opening in the remaining parts of the dressing corresponding to the marked contour of the wound bed is cut, for example by scissors. Such cutting is facilitated by the hole 9 in the dressing, which has dimensions making the introduction of a pair of scissors through this opening easy for the user. The diameter of hole 9 can be 5-10 mm. The cut is made through all the layers of dressing 1 remaining after the removal of protection layer 6.

The cut part of the dressing 1 is then removed. Thereby, a dressing having an inner opening with a contour corresponding to the contour of the wound bed is created.

The release layer 5 is then removed and the dressing 1 with its cut opening is thereafter applied to the skin surrounding the wound so that the edges of the opening cut therein correspond with the contour of the wound. In order to facilitate removal of the release layer 5, this layer is provided with grip tabs 12,13 corresponding in size and horizontal placing with grip tabs 10, 11 of the stiffening layer 4. The stiffening layer 4 has a somewhat greater adhesion to the film layer 2 (via adhesive layer 8) than the adhesion of the release layer 5 to the adhesive layer 3 so if tabs 10 and 12 are held onto while moving these tabs away from each other, this will result in removal of the release layer 5.

Finally, the stiffening layer 4 is removed from the film layer 2. In the embodiment shown in FIGS. 1 and 2, a cutting line 14 through the stiffening layer 4 divides this layer into a central portion 16 and a frame portion 17. In order to facilitate the application of the dressing 1 to the skin, the remaining part of the central portion 16 can optionally be taken away before the step of applying the dressing to the skin around a wound. The frame portion 17 remaining attached to the film layer 2 will facilitate handling of the dressing during the application step. After application, the frame portion 17 will also be taken away. One or more slits 15 can be made in the frame portion 17 in order to make the removal of the portion 17 in one or more pieces easy.

Since the opening cut out of the dressing 1 exactly corresponds to the outer contour of the wound, dressing 1 makes it possible to accurately protect all skin surrounding a wound so that no maceration, irritation or damage to the skin will occur neither during use of the dressing 1 or when this is removed to be changed or removed for other reasons. The film layer 2 provides a perfect surface for the fixation of dressings for the wound and possible portions of the dressing 1 projection outside such fixation means will provide a non tacky outer surface.

The plastic film 2 is preferably of polyurethane and has a thickness of 10-100 μm, preferably 10-50 μm. Other plastic materials such as polyethylene, polypropylene or polyester could also be used.

A principal function of the adhesive coating 3 is to attach the dressing 1 tightly to the skin of the patient, so that the fluid-borne transport of bacteria between the skin and the adhesive coating is prevented, and to attach the dressing securely to the skin, so that the product remains in place during all the normal loadings to which dressings are subjected.

The adhesive in the coating must also be skin friendly and must permit removal of the film dressings without causing damage to the skin or pain to the patient. This requirement presents a major problem in the case of those types of pressure-sensitive adhesives that are currently used as adhesive coatings for film dressings. Such adhesives often attach themselves to the skin so strongly that parts of the Stratum Corneum, that is to say the uppermost layer of the skin, become stuck to the adhesive and are pulled away from the skin when the film dressing is loosened. This can lead to irritation of and damage to the skin.

An example of a skin-friendly adhesive suitable to be used in the present invention is a tacky silicone gel. Silicone gel is very soft and possesses low surface energy, and it adapts very well to the skin, that is to say it flows out into any unevennesses in the skin and creates a large contact surface between the skin and the silicone gel. This large contact surface helps the silicone gel to become attached securely to the skin, in spite of the fact that the strength of the adhesive attachment of the silicone gel to the skin is not in itself so strong. The adhesive strength constitutes a measure of the energy required in order to separate/pull off the adhesive layer from the skin. A contributory factor to the fact that high energy, and thus a high pulling force, is required in order to remove the silicone gel from the skin, in spite of the relatively weak strength of the adhesive attachment, is that a lot of energy is consumed in stretching the soft silicone gel before it releases from the skin. The softer and thicker the layer of silicone gel, the greater the force/energy required to remove the gel from the skin.

The use of a harder adhesive will require a stronger strength of adhesive attachment in order for the pulling force to be as high as for a softer adhesive. A strong adhesive attachment between the skin and the adhesive can easily lead to skin cells being pulled from the skin in conjunction with the removal of the adhesive.

Another disadvantage associated with harder adhesives is that these are capable of flowing outwards eventually and thus increasing the size of the contact surface with the skin, with the result that the pulling force eventually increases, which can lead to such adhesives eventually becoming difficult to remove from the skin. Unlike harder adhesives, softer adhesives such as silicone gels achieve their full adhesive strength all at once so that their pulling force remains constant as time passes.

Suitable soft, skin friendly adhesives for the adhesive layer 3 are for example addition curing RTV (Room Temperature Vulcanizing) silicone systems which after mixing crosslink and form a self-adhering gel. Examples of such RTV silicone systems are given in EP 0 300 620 A1 describing so called “gel forming compositions” constituted of a alkenylsubstituted polyorganosiloxane containing hydrogen atoms bonded to some of the silicon atoms and a platinum catalysator.

Wacker SilGel 612 is a commercially available RTV-silicone system. It is a two component system. By varying the proportions between the two components A:B from 1.0:0.7 to 1.0:1.3 the softness and adhesion level of the formed silicone material can be varied.

Examples of further soft, skin friendly silicone gels that can be used are NuSil MED-6340, NuSil MED3-6300, NuSil MED12-6300 from NuSil Technology, Carpintiera, Ga., USA and Dow Corning 7-9800 from Dow Corning Corporation, Midland, USA.

Also other soft, skin friendly adhesives might be used in the present invention, for example hot-melts similar to Dispomelt® 70-4647 from National Starch and Chemical Company, Bridgewater, N.J., USA.

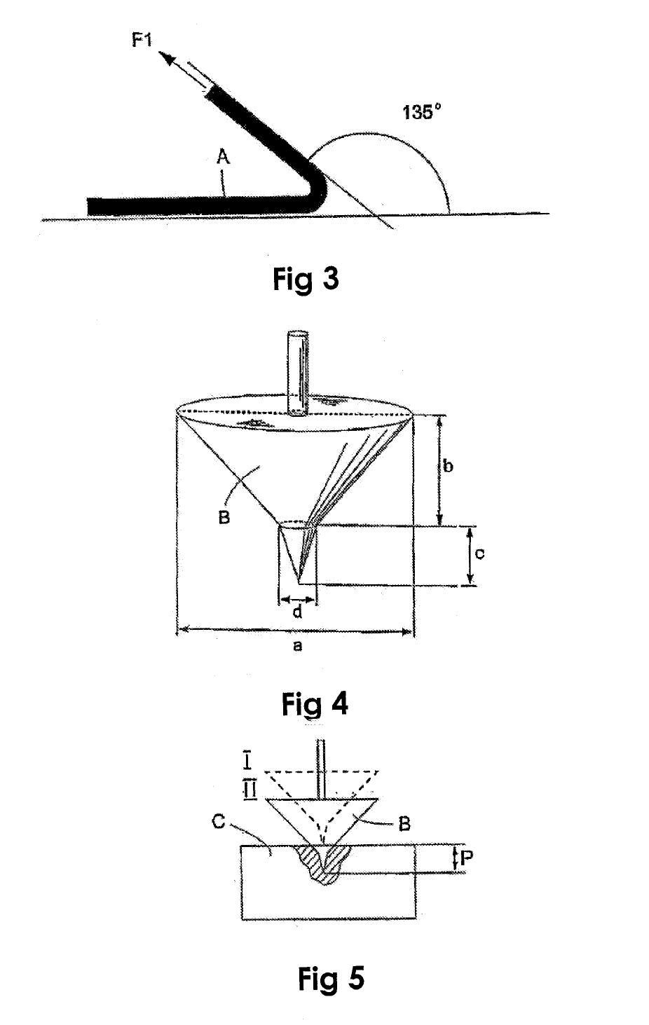

Because the characteristics of the skin vary from person to person, the adhesive capacity of the adhesive coating to the skin naturally also varies for different patients. The adhesive strength is also dependent on the thickness of the soft adhesive and the mechanical characteristics of the barrier layer. The standard methods for the measurement of adhesion that are available today use plates of various kinds, for example made of steel or glass, and they do not produce values that are relevant for the measurement of the adhesion to the skin. The values for the strength of the adhesive attachment of an adhesive to the skin, as indicated below, must be measured by means of a method of the kind illustrated schematically in FIG. 3 and developed by the applicants.

Strips of a self-adhesive dressing, for which the strength of the adhesive attachment to the skin is to be measured, are punched out with dimensions of 25×125 mm. It should be noted that all the strips are also provided with a carrier layer on the rear side of the film dressing. (The function of this carrier layer is to stiffen up the strips when they are applied to the skin). The strips are then applied to the skin on the back of healthy volunteers. The strips are carefully smoothed into place with a finger, and the carrier layer on the rear side of the strips is then removed. Finally, the strips are pressed securely against the skin for 3 seconds with the help of a sponge made of foam plastic (42×182 mm, thickness=48 mm) glued securely to a steel sheet (50×200 mm, thickness=1 mm). The applied pressure is estimated to 6 kN/m². The strips are left in place on the skin for 2 minutes. The strips are then pulled off at a rate of 25 mm/sec, and the removal force F1 is measured. The angle of removal, that is to say the obtuse angle that is formed between the surface of the skin and the removed part of the strip, must be 135°. The strength of the adhesive attachment of the strip to the skin is constituted by the mean value of the force F1.

Adhesives that are suitable for use in film dressings in accordance with the invention must exhibit a strength of adhesive attachment of at least 0.2 N/25 mm in accordance with this method. The strength of adhesive attachment is preferably 1-2.5 N/25 mm.

Adhesives in accordance with the present invention must exhibit a softness that exceeds 10 mm measured by means of a method based on ASTM D 937 and ASTM D 51580. Certain deviations, as can be appreciated below, have been made. FIGS. 4 and 5 illustrate this modified method of measuring the softness of an adhesive by causing a cone B with a weight of 62.5 g to penetrate down by the effect of gravity into a 30 mm thick test piece C of the adhesive for which the softness is to be determined. The test piece is obtained by filling a cylindrical glass container having an internal diameter of 60 mm and an internal height of 35-40 mm, with adhesive to a depth of 30 mm. In the case of a silicone gel, it is necessary to fill a non-cured silicone prepolymer into the container, and then to cross-link it to a gel in the glass cylinder. The cone used is illustrated in FIG. 4 and has the following dimensions: a=65 mm, b=30 mm, c=15 mm and d=8.5 mm. In the performance of the method for measurement of the softness, the cone B is first lowered down into a position I, as illustrated with broken lines in FIG. 5, and in which the tip of the cone just touches the surface of the test piece C. The cone B is then released, so that it is able to penetrate down into the test piece C by the effect of gravity. The number of millimetres by which the tip B of the cone has penetrated into the test piece C after 5 seconds is measured and constitutes the penetration value P, the value of which is greater in proportion to the softness of the test piece. The penetration value P represents the softness index used in the present invention. A PNR 10 penetrometer supplied by Sommer & Runge KG, Germany is used in the performance of the method.

It has also been found that the risk for leakage due to irregularities in the skin will be less the softer the adhesive and the higher the basis weight. It is therefore preferred that the softness of the adhesive is greater than 10 mm and the basis weight is at least 50 g/m².

In order to ensure that only a low application force is required in conjunction with the application of film dressings in accordance with the present invention, it is recommended that the softness of the soft, skin friendly adhesive that is used should be greater than 10 mm, and that it should preferably lie between 12 and 17 mm. The softer an adhesive, the more rapidly it will flow into any unevennesses in the substrate, which means that the dressings including such adhesives are leakproof immediately after their application to normal skin. At a softness value greater than 17 mm, there is a risk of the inner cohesion of the adhesive being too low, so that residues of adhesive are left behind on the skin in conjunction with the removal of an applied dressing.

Furthermore, no hairs are pulled out when removing film dressings provided with soft, skin friendly adhesive.

The products proposed in the present invention are normally supplied packed in sterile conditions, which means that the adhesives used must be capable of being sterilized, as must other components of such articles, of course.

The stiffening frame 4 is made of a plastic film of a suitable thickness, for example a polypropylene having a thickness of 20-200 μm, preferably 40-100 μm. Other plastic materials such as polyethylene, polystyrene, polyester and dimethylsiloxane can also be used.

The release layer 5 is constituted by a plastic film, for example of polyethylene, having protrusions on the side thereof facing the adhesive layer 3.

The protection layer can be made of any transparent material but is preferably made of a plastic material, such as polyethylene.

In the embodiment shown in FIGS. 1 and 2, the protection layer 6 is attached to the release layer 5 by a string of adhesive 7 which is extended between opposite edges of the release layer 5 without reaching those edges. More than one strings of adhesive can be used but the attachment between should be weak so the protection layer is easy to separate from the release layer 5. The strings can be continuous or discontinuous, for example consist of a pattern of glue spots. It is also possible to use one or more weld seams instead of adhesive seams. It is to advantage if at least one region along at least one edge of the release layer is unattached to the protection layer.

In order to supervise the development of wound healing, the contour of the wound at the time of cutting the dressing 1 can be registered by saving and storing the cut out part of the central portion 16 of the stiffening layer 4 or the cut out part of a stiffening layer lacking a line cutting the stiffening layer into a frame portion and a central portion. It is of course also possible to save the part of the stiffening layer remaining after cutting and containing an opening having the shape corresponding to the contour of the wound instead of the part cut out of the stiffening layer. If the release layer consists of a one piece layer which is usually not the case, the cut out part thereof can be saved instead of the cut out part of the stiffening layer.

In the embodiment disclosed in FIG. 2A the stiffening layer 4 is releasably attached to the film layer 2 by an adhesive layer 8 but other ways of releasably attaching layer 4 to layer 2 can be used, the layers can for example be coextruded onto each other. In FIG. 2B is a film dressing 1 according to a second preferred embodiment disclosed in a view corresponding to FIG. 2A. The components in the dressing 1 according to FIG. 2B are identical to the components in the embodiment shown in FIG. 2A, the only difference being that the adhesive layer 8 is lacking in the embodiment according to FIG. 2B. The same reference numerals are therefore used in both embodiments. In the embodiment shown in FIG. 2B, the stiffening layer 4 is attached to the film layer 2 by application of heat and pressure.

The dressing according to the invention is thus easy to adapt to precisely cover and protect the skin around a wound and is also easy to apply. Another advantage of this dressing is that the plastic film is a suitable base for affixing medical devices, such as wound pads or wound dressings, by a self-adhering adhesive. It is especially advantageous to affix an absorbent wound dressing having self-adhering edges so that it covers the wound bed to the protective dressing according to the present invention.

In this connection, a dressing with an aggressive self-adhering adhesive having a large adhesive force ensuring a strong adhesive attachment can be chosen without risk for the aggressive adhesive to be affixed to the skin and cause damage to the skin when the dressing is removed. When such a dressing is affixed to the protective dressing according to the present invention, the adhesion between the wound dressing (or other type of medical device) and the upper side of the protective dressing according to the invention is stronger than the adhesion between the dressing according to the invention and the skin surrounding the wound so that when removing the wound dressing the protective dressing will be stuck to the wound dressing and be removed together with the wound dressing. Since the dressing according to the present invention is provided with a skin friendly adhesive, the risk for damaging the skin when removing a wound dressing together with the protective dressing according to the present invention is small. If non-adhesive wound dressings or pads are used together with a protective dressing according to the present invention, such dressings can be affixed thereto with the aid of self-adherent tapes.

It is of course also advantageous to temporarily affix medical devices, such as a wound dressing, to the upper side of the protective dressing according to the present invention. In such a case, a wound dressing (or other type of medical device) coated with an adhesive having a lower adhesive force to the plastic film than the adhesive force with which the protective dressing is attached to the skin, should be chosen. Thereby, the wound dressing could be removed without dislodging the protective dressing according to the invention. This way of affixing a wound dressing is especially advantageous for wound dressings that are to changed often. Since the protective dressing according to the present invention will remain attached to the skin during every change of wound dressing affixed thereto, the changing of wound dressing can be made without any risk to damage of the skin. If non-adhesive wound dressings or pads are used together with a protective dressing according to the present invention, such dressings can be affixed thereto with the aid of self-adherent tapes having an adhesive with a lower adhesion to the plastic film than the adhesion to the skin of the protective dressing.

The shown embodiments of the present invention can of course be modified without leaving the scope of invention. For example can the shape of the dressing be different from the shape shown, for example circular. Moreover, the stiffening layer can be in one piece, i.e. without cutting line. The hole in the dressing need not be circular but can have other shape, for example rectangular, square, oval, etc. Furthermore, the protection layer need not be larger than the rest of the dressing even if this is preferred, it is even possible that the protection layer is smaller as long as it covers a wound in use. In the shown embodiment the seam attaching the protection layer to the release layer runs parallel to the edge of the release layer containing the grip tab but can of course also run along an edge perpendicular thereto. The protection layer could also be attached to the release layer by a pattern of glue or weld spots, which preferably leaves one or all the edge portions of the release layer unattached to the protection layer. The frame portion of the stiffening layer can be divided into several parts by cutting lines. The scope of invention shall therefore only be restricted by the wording of the enclosed patent claims.

1. A transparent film dressing (1) for protection of skin surrounding a wound, said dressing comprising a film layer (2) coated with a skin friendly adhesive (3) on one side, a stiffening layer (4) releasably attached to the film layer on the side thereof opposite to the side coated with adhesive, and a release layer (5) releasably attached to the adhesive coating on the film layer, characterised in that a protection layer (6) is releasably attached to the release layer (5) on the side thereof opposite to the side attached to the adhesive coating (3), and in that a hole (9) extends through the stiffening layer (4), the coated film layer (2,3) and the release layer (5) but not through the protection layer, all said layers being transparent.

2. The transparent film dressing (1) according to claim 1, wherein the protection layer (6) at least in part or parts extends beyond the contour of the other layers (4,2,3,5) of the dressing (1).

3. The transparent film dressing (1) according to claim 2, wherein the protection layer (6) is attached to the release layer by one or more seams (7).

4. The transparent film dressing (1) according to claim 3, wherein the protection layer is attached to the release layer by one seam (7) extending between opposite edges of the of the release layer (13) without reaching said edges, said seam being located in an end part of the release layer but distanced from an adjacent end edge.

5. The transparent film dressing (1) according to claim 3, wherein each of said seams is discontinuous.

6. The transparent film dressing (1) according to claim 3, wherein each said seam is constituted by a string (7) of adhesive.

7. The transparent film dressing (1) according to claim 3, wherein each said seam is constituted by a weld seam.

8. The transparent film dressing (1) according to claim 1, wherein the protection layer (6) is a plastic film and the release layer (5) is a plastic film having a pattern of protrusions on each of its sides.

9. The transparent film dressing (1) according to claim 1, wherein the stiffening layer (4) is divided into a frame portion (17) and a central portion (16) by a cutting line (14).

10. Method of applying a film dressing (1) according to claim 1 to the skin surrounding a wound comprising the following steps;
applying the dressing (1) over a wound so that the wound bed and an area surrounding the wound is covered by the film layer (2) of the dressing and so that the hole (9) in the dressing is located over the wound bed,
marking the contour of the wound bed on the stiffening layer (4),
removing the dressing (1) from the wound,
removing the protection layer (6) from the dressing (1),
cutting an opening in the remaining parts of the dressing (1) corresponding to the marked contour of the wound bed, whereby the cutting is facilitated by the hole (9) in the dressing,
removing the cut part of the dressing (1),
removing the release layer (5),
applying the remaining part of the dressing (1) over the wound so that the edges of the opening cut therein correspond with the contour of the wound, and
removing the stiffening layer (4) from the film layer (2).

11. Method according to claim 10 for applying a film dressing comprising a film layer (2) coated with a skin friendly adhesive (3) on one side, a stiffening layer (4) divided into a frame portion (17) and a central portion (16) by a cutting line (14), and releasably attached to the film layer on the side thereof opposite to the side coated with adhesive, a release layer (5) releasably attached to the adhesive coating on the film layer, a protection layer (6) releasably attached to the release layer (5) on the side thereof opposite to the side attached to the adhesive coating (3), and a hole (9) extending through the stiffening layer (4), the coated film layer (2,3) and the release layer (5) but not through the protection layer, all said layers being transparent, wherein the remaining part of the central portion (16) of the stiffening layer (4) is removed before the step of applying the remaining part of the dressing (1) over the wound.

12. Method according to claim 10, wherein the cut out portion of the stiffening layer (4) is saved in order to document the size of the wound bed at the time of application of the dressing (1).

13. Method according to claim 10, wherein the portion of the stiffening layer (4) remaining after the cutting out of a portion of the dressing (1) corresponding to the size of the wound bed is saved in order to document the size of the wound bed at the time of application of the dressing.

14. Method according to claim 11, wherein the remaining part of the central portion (16) of the stiffening layer (4) is saved in order to document the size of the wound bed at the time of application of the dressing (1).
